(19) United States
(12) Patent Application Publication
(10) Pub. No.: US 2010/0075899 A1
(43) Pub. Date: Mar. 25, 2010

(54) TARGETED PHOTODYNAMIC THERAPY AGENT

(75) Inventors: Gang Zheng, Toronto (CA); Klara Stefflova, Philadelphia, PA (US)

Correspondence Address: Pearl Cohen Zedek Latzer, LLP, 1500 Broadway, 12th Floor, New York, NY, 10036, US

(73) Assignees: Juan CHEN, Toronto, ON (CA); TRUSTEES OF THE UNIVERSITY OF PENNSYLVANIA, Philadelphia, PA (US)

(21) Appl. No.: 12/443,019

(22) PCT Filed: Sep. 28, 2007
(86) PCT No.: PCT/US07/79922

§ 371 Date: Nov. 30, 2009

Related U.S. Application Data
(60) Provisional application No. 60/847,836, filed on Sep. 28, 2006.

(51) Int. Cl. A61K 38/16; C07K 5/113; C07K 5/10; C07K 7/06; C07K 7/08; C07K 14/00; C12N 5/09; A61K 38/07; A61K 38/08; A61K 38/10; A61P 35/00

(52) U.S. Cl. 514/12; 530/330; 530/328; 530/324; 530/325; 530/326; 530/327; 435/325; 514/18; 514/17; 514/16; 514/15; 514/14; 514/13

(57) ABSTRACT

The present invention relates to the field of conjugates comprising a substrate, a photosensitizer, and a targeting ligand, and methods for their use. More particularly, the invention further relates to methods for treating cancer in a subject using the conjugates of the present invention.

### FIELD OF THE INVENTION

The present invention relates to the field of targeted photodynamic therapy agents. The invention further relates to compositions and methods for treating disease such as cancer in a subject using the photodynamic therapy agents of the present invention.

### BACKGROUND

When a porphyrin-like molecule is activated by light, it relaxes to its ground state primarily in two ways it emits a photon or transfers the energy, producing singlet oxygen (Milgron, L. R. (1997) The Colours of Life. Chapter 3: How do they do it?—Making oxygen (Milgron, L. R. ed.) pp. 65-110, Oxford University Press Inc., New York; Roeder, B., Naether, D., Lewald, T., Braune, M., Nowak, C. and Freyer, W. (1990) Photophysical properties and photodynamic activity in vivo of some tetrapyrroles. Biophys. Chem. 35, 303-312.). The detectable outcomes are fluorescence (Licha, K. (2002) Contrast Agents for Optical Imaging. Top. Curr. Chem. 222, 1-29.) and phototoxicity (Oleinick, N. L. and Evans, H. H. (1998) The photobiology of photodynamic therapy: cellular targets and mechanisms. Radiat. Res. 150, S146-156.), making the porphyrin-based photosensitizer (PS) a perfect candidate for image-guided therapy.

The success of cancer treatment is often short-term owing to the difficulty in clearing-out all the cancer cells. Being able to clearly identify the cancer cells shortly before or during the treatment would most likely increase the success of the therapy, making imaging and therapy a beneficial union (Bogaards, A., Varma, A., Collens, S. P., Lin, A., Giles, A., Yang, V. X., Bilbao, J. M., Lilge, L. D., Muller, P. J. and Wilson, B. C. (2004) Increased brain tumor resection using fluorescence image guidance in a preclinical model. Lasers Surg. Med. 35, 181-190; Coleman, C. N. (2003) Linking radiation oncology and imaging through molecular biology (or now that therapy and diagnosis have separated, it's time to get together again!). Radiology 228, 29-35; Huang, X., El-Sayed, I. H., Qian, W. and El-Sayed, M. A. (2006) Cancer cell imaging and photothermal therapy in the near-infrared region by using gold nanorods. J. Am. Chem. Soc. 128, 2115-2120; and Dougherty, T. J. (1987) Photosensitizers: therapy and detection of malignant tumors. Photochem. Photobiol. 45, 879-889.). The utilization of the natural connection of near-infrared (NIR) fluorescence imaging—a sensitive and accessible means of in vivo cancer detection (Licha, K. (2002) Contrast Agents for Optical Imaging. Top. Curr. Chem. 222, 1-29; Gurfinkel, M., Thompson, A. B., Ralston, W., Troy, T. L., Moore, A. L., Moore, T. A., Gust, J. D., Tatman, D., Reynolds, J. S., Muggenburg, B., Nikula, K., Pandey, R., Mayer, R. H., Hawrysz, D. J. and Sevick-Muraca, E. M. (2000) Pharmacokinetics of ICG and HPPH-car for the detection of normal and tumor tissue using fluorescence, near-infrared reflectance imaging: a case study. Photochem. Photobiol. 72, 94-102; and Moon, W. K., Lin, Y., O'Loughlin, T., Tang, Y., Kim, D. E., Weissleder, R. and Tung, C. H. (2003) Enhanced tumor detection using a folate receptor-targeted near-infrared fluorochrome conjugate. Bioconjug. Chem. 14, 539-545)—with photodynamic therapy (PDT)—a promising cancer therapy using a laser to excite a tumor-associated photosensitizer that produces short lived cytotoxic singlet oxygen (Dougherty, T. J., Gomer, C. J., Henderson, B. W., Jori, G., Kessel, D., Korbelik, M., Moan, J. and Peng, Q. (1998) Photodynamic therapy. J. Natl. Cancer Inst. 90, 889-905; and Niedre, M. J., Secord, A. J., Patterson, M. S, and Wilson, B. C. (2003) In vitro tests of the validity of singlet oxygen luminescence measurements as a dose metric in photodynamic therapy. Cancer Res. 63, 7986-7994) is discussed herein. Although many target specific NIR imaging and PDT agents are being developed, the common limit remains: these agents are mostly lost in organs involved in drug clearance, generating an unwanted toxicity and elevated background (Joni, G. (1996) Tumour photosensitizers: approaches to enhance the selectivity and efficiency of photodynamic therapy. J. Photochem. Photobiol., B 36, 87-93).

There have been many attempts to enhance the photosensitizer's efficacy by targeting “cancer fingerprints” (Hanahan, D. and Weinberg, R. A. (2000) The hallmarks of cancer. Cell 100, 57-70) through association with various vehicles (Sharman, W. M., van Lier, J. E. and Allen, C. M. (2004) Targeted photodynamic therapy via receptor mediated delivery systems. Adv. Drug Delivery Rev. 56, 53-76; and Konan, Y. N., Gurny, R. and Allemann, E. (2002) State of the art in the delivery of photosensitizers for photodynamic therapy. J. Photochem. Photobiol., B 66, 89-106) like proteins (e.g. BSA targeting scavenger receptors on macrophages (Hamblin, M. R., Miller, J. L. and Ortel, B. (2000) Scavenger-receptor targeted photodynamic therapy. Photochem. Photobiol. 72, 533-540), transferrin (Derycke, A. S., Kamuhabwa, A., Gijsens, A., Roskams, T., De Vos, D., Kasran, A., Huwyler, J., Missiaen, L. and de Witte, P. A. (2004) Transferrin-conjugated liposome targeting of photosensitizer AlPcS4 to rat bladder carcinoma cells. J. Natl. Cancer Inst. 96, 1620-1630) or LDL (Zheng, G., Chen, J., Li, H. and Glickson, J. D. (2005) Rerouting lipoprotein nanoparticles to selected alternate receptors for the targeted delivery of cancer diagnostic and therapeutic agents. Proc. Natl. Acad. Sci. U.S.A. 102, 17757-17762)), tumor-selective monoclonal antibodies (Morgan, J., Gray, A. G. and Huehns, E. R. (1989) Specific targeting and toxicity of sulphonated aluminium phthalocyanine photosensitized liposomes directed to cells by monoclonal antibody in vitro. Brit. J. Cancer 59, 366-370), saccharides (Chen, X., Hui, L., Foster, D. A. and Drain, C. M. (2004) Efficient synthesis and photodynamic activity of porphyrin-saccharide conjugates: targeting and incapacitating cancer cells. Biochemistry 43, 10918-10929), aptamers (Farokhzad, O. C., Karp, J. M. and Langer, R. (2006) Nanoparticle-aptamer bioconjugates for cancer targeting. Expert Opin. Drug Delivery 3, 311-324) or other small molecule ligands (e.g. short peptides or peptidomimetics) (Rosenkranz, A. A., Jans, D. A. and Sobolev, A. S. (2000) Targeted intracellular delivery of photosensitizers to enhance photodynamic efficiency. Immunol. Cell Biol. 78, 452-464; Ichikawa, K., Hikita, T., Maeda, N., Yonezawa, S., Takeuchi, Y., Asai, T., Namba, Y. and Oku, N. (2005) Antiangiogenic photodynamic therapy (PDT) by using long-circulating liposomes modified with peptide specific to angiogenic vessels. Biochim. Biophys. Acta 1669, 69-74; Renno, R. Z., Terada, Y., Haddadin, M. J., Michaud, N. A., Gragoudas, E. S, and Miller, J. W. (2004) Selective photodynamic therapy by targeted verteporfin delivery to experimental choroidal neovascularization mediated by a homing peptide to vascular endothelial growth factor receptor-2. Arch. Opthalmol. 122, 1002-1011; and Bisland, S. K., Singh, D. and Gariepy, J. (1999) Potentiation of chlorin e6 photodynamic activity in vitro with peptide-based intracellular vehicles. Bioconjug. Chem. 10, 982-992). By attaching Pyro to Folate, an easy-to-conjugate, small, soluble, and non-immunogenic tumor homing molecule (Hilgenbrink, A. R. and Low, P. S. (2005) Folate receptor-mediated drug targeting: from therapeutics to diagnostics. J. Pharm. Sci. 94, 2135-2146) targeting cancer cells overexpressing FR (Moon, W. K., Lin, Y., O′Loughlin, T., Tang, Y., Kim, D. E., Weissleder, R. and Tung, C. H. (2003) Enhanced tumor detection using a folate receptor-targeted near-infrared fluorochrome conjugate. Bioconjug. Chem. 14, 539-545; Kukowska-Latallo, J. F., Candido, K. A., Cao, Z., Nigavekar, S. S., Majoros, I. J., Thomas, T. P., Balogh, L. P., Khan, M. K. and Baker, J. R., Jr. (2005) Nanoparticle targeting of anticancer drug improves therapeutic response in animal model of human epithelial cancer. Cancer Res. 65, 5317-5324; and Schneider, R., Schmitt, F., Frochot, C., Fort, Y., Lourette, N., Guillemin, F., Muller, J. F. and Barberi-Heyob, M. (2005) Design, synthesis, and biological evaluation of folic acid targeted tetraphenylporphyrin as novel photosensitizers for selective photodynamic therapy. Bioorg. Med. Chem. 13, 2799-2808), we expected to enhance the cancer-specificity of Pyro. To potentiate this specificity, we have inserted a short peptide sequence to serve as a spacer, a solubilizer, and a pharmacomodulator (Schottelius, M., Rau, F., Reubi, J. C., Schwaiger, M. and Wester, H. J. (2005) Modulation of pharmacokinetics of radioiodinated sugar-conjugated somatostatin analogues by variation of peptide net charge and carbohydration chemistry. Bioconjug. Chem. 16, 429-437). As a spacer, it makes the Folate more accessible to FR and, being small and hydrophilic, it decreases its retention in the excretion organs making it more suitable for in vivo applications.

The present invention describes a receptor-targeted, water soluble, and pharmacomodulated photodynamic therapy (PDT) agent that selectively detects and destroys the targeted cancer cells while sparing normal tissue. This was achieved by minimizing the normal organ uptake (e.g., liver and spleen) and by discriminating between tumors with different levels of receptor expression.

### SUMMARY OF THE INVENTION

In one aspect, the present invention is directed to a conjugate comprising a hydrophilic substrate, a photosensitizer comprising a fluorophore, and a targeting ligand.

In another aspect, the invention provides a method for treating a disease state comprising the steps of contacting diseased tissue with the conjugates of the present invention and exposing the diseased tissue to an effective amount of artificial irradiation. In one embodiment the disease state is cancer.

### BRIEF DESCRIPTION OF THE DRAWINGS

FIG. 1 Pyro-GDEVDGSGK-Folate (SEQ ID NO: 10) comprises three principal components: 1) Pyropheophorbide a is an imaging (NIR fluorescence) and therapeutic (photosensitizer for PDT) agent; 2) peptide sequence as a stable and hydrophilic linker with the possibility of exchanging it for various organelle targeting sequences; and 3) folate as a cancer-specific delivery vehicle.

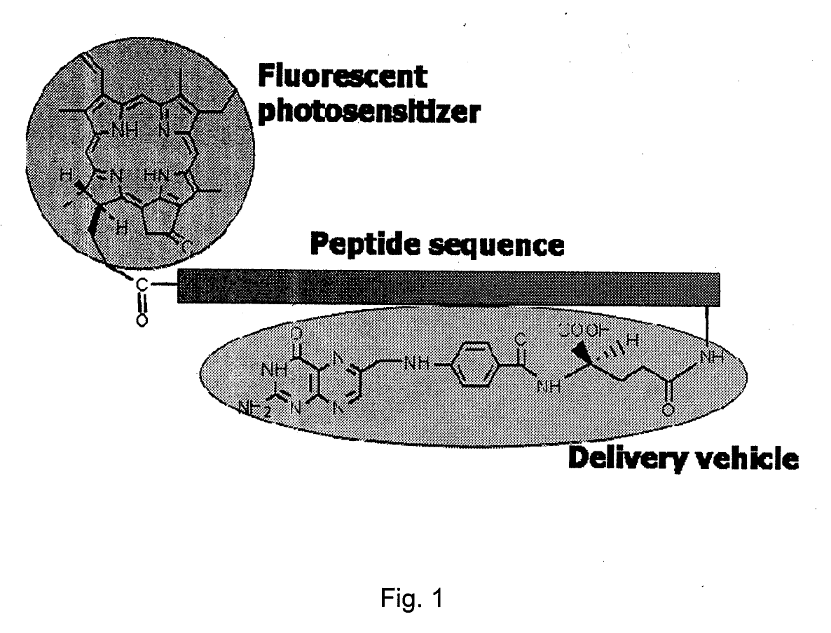

FIG. 2 Synthesis (A), MALDI-ToF (B) and HPLC (C) of Pyro-GDEVDGSGK-Folate.

FIG. 3 A) KB cells alone; B) KB cells incubated with 40 μM of PPF for 5 hours; C) HT 1080 cells alone; and D) HT 1080 cells incubated with 40 μM of PPF for 5 hours. KB cells overexpressing folate receptor uptake PPF better than HT 1080 cells that are folate receptor negative. Pyro-K-Folate, lacking the peptide sequence, is c) taken up into the KB cells (FR+) less efficiently (50%) than Pyro-GDEVDGSGK-Folate d) HT 1080 cells and CHO cells (both FR−) uptake PPF and PKF with the same efficiency (flow cytometry results)—peptide is therefore useful as a spacer (making the folate more accessible for more efficient delivery) and hydrophilic component (for increasing water solubility).

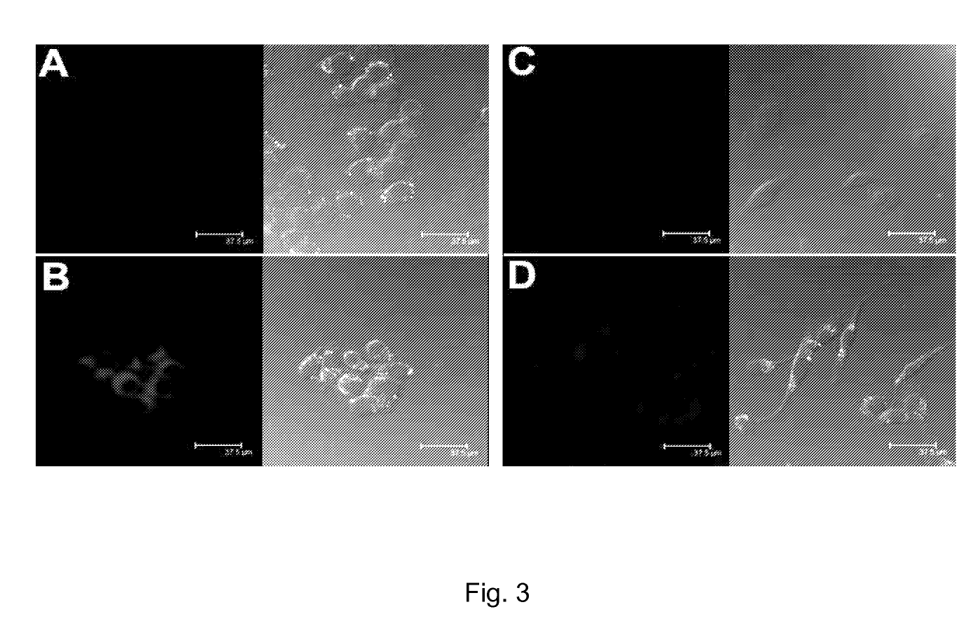

FIG. 4 Pyro-K-Folate, lacking the peptide sequence, is: a) more hydrophobic (HPLC-left) and less soluble in water.

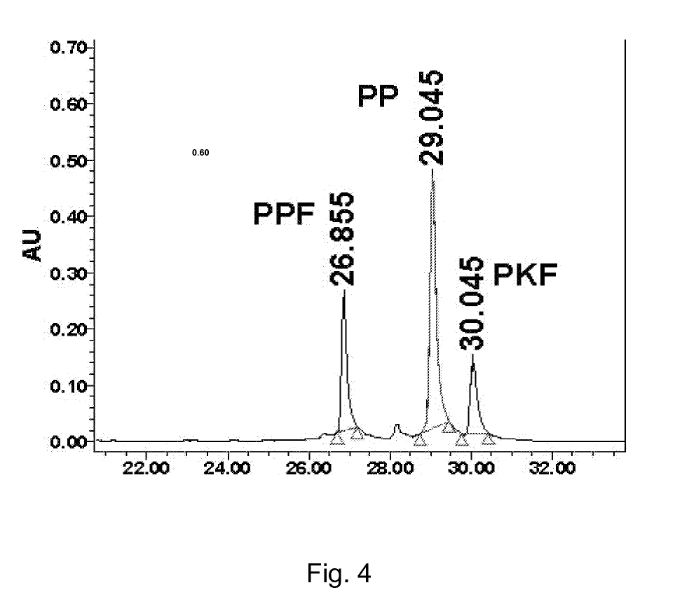

FIG. 5 Viability of KB cells (FR+, cancer cells), HT 1080 cells (FR−, cancer cells) and CHO cells (FR−, normal cells) incubated with 0.5, 5, and 15 μM of PPF and treated with light (1, 5 or 10 J/cm²) or kept in dark (0 J/cm²). KB cells have reduced viability below 10% when incubated with 5 μM PPF and treated with the lowest light dose (1 J/cm²), but CHO cells reach this level of cell death for the same concentration of PPF drug only when treated with the highest light dose (10 J/cm²). In case of HT 1080, even the highest drug dose (15 μM) and light dose (10 J/cm²) didn't effectively reduce the viability below 10%.

FIG. 6 More than half (56%) of the KB cells incubated with 5 μM PPF and treated with 5 J/cm² light dose (B) show hypergranular/shrunken morphology typical for apoptotic cells when compared to cells treated with PPF only (A) that have only 3% of these non-viable cells. These cells also show 10 times higher staining with fluorescently labelled antibody against active caspase-3, suggesting that for this concentration and light dose, the major cell death is via apoptosis.

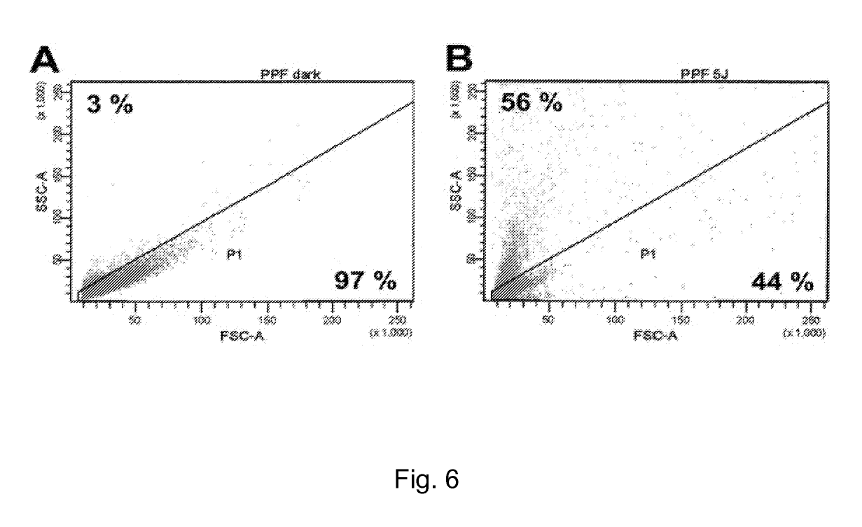

FIG. 7 Monitoring fluorescence signal distribution after intravenous injection of three different doses of PPF to double tumor bearing mice that have KB tumor (FR+) on the right side and HT 1080 tumor (FR−) on the left side, using a Xenogen imager. A) Prescan; B) 5 minutes after injection of 50 nmol (left mouse), 100 nmol (middle mouse) and 150 nmol (right mouse) of PPF, there is an immediate uptake in both tumors (slightly higher for HT 1080, possibly due to more developed vasculature); C) 6 hours after injection; and D) 24 hours after injection. At this point it is clear that PPF preferentially accumulates in KB tumor.

FIG. 8 Xenogen images monitoring the biodistribution of intravenously administered Pyro-GDEVDGSGK (SEQ ID NO: 10, PP, no targeting) and Pyro-GDEVDGSGK-Folate (SEQ ID NO: 10, PPF, targeting folate receptor overexpressing KB tumor) in double tumor mouse (left-HT 1080, right KB tumor). A) prescan; B) 2 minutes after iv injection of PPF (left mouse) and PP (right mouse); C) 15 minutes after iv; D) 1 hour 45 minutes after iv; E) 6 hours 15 minutes after iv; and F) organs dissected from the mouse 30 hours after iv, rows 1 and 2 contain organs from PPF mouse, rows 3 and 4 contain organs from PP mouse. Images of both the living mice and dissected organs show significantly higher accumulation of PPF in KB tumor than in HT 1080 tumor but similar distribution of non-targeting PP in both tumors.

FIG. 9 Quantitation of PPF and PP uptake in dissected organs showing the highest fluorescence in KB tumors of PPF treated mice. The data are normalized with the respect to muscle uptake.

FIG. 10 Folate receptor positive and negative mice treated with targeted PDT agent with built-in apoptosis reporter. Mouse #1: KB tumor (Folate receptor positive); Mouse #2: HT 1080 tumor (Folate receptor negative); A: 2.5 h post iv injection, before PDT; B: 5 h post iv injection, 2 h after PDT.

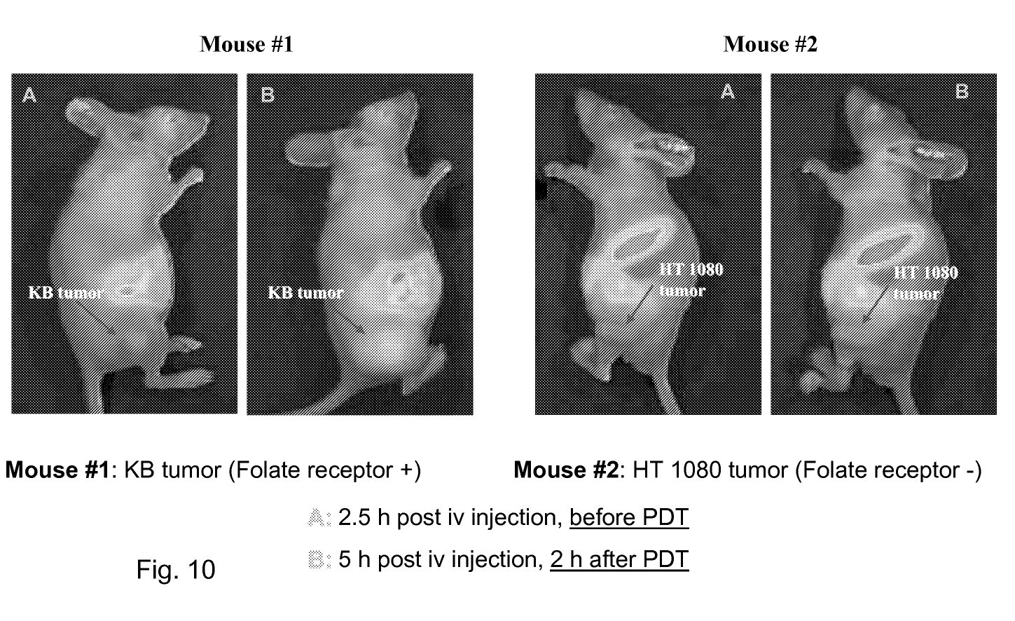

FIG. 11 Diagram depicting a conjugate which is an activatable PDT agent of the present invention, and an apoptosis reporter. “PS” is the photosensitizer which generates reactive oxygen species (ROS), such as singlet oxygen (¹O₂) or superoxide free radicals; “BHQ” is the quencher which quenches the fluorescence of the photosensitizer when the photosensitizer and quencher are in close proximity. “Folate” is the targeting ligand.

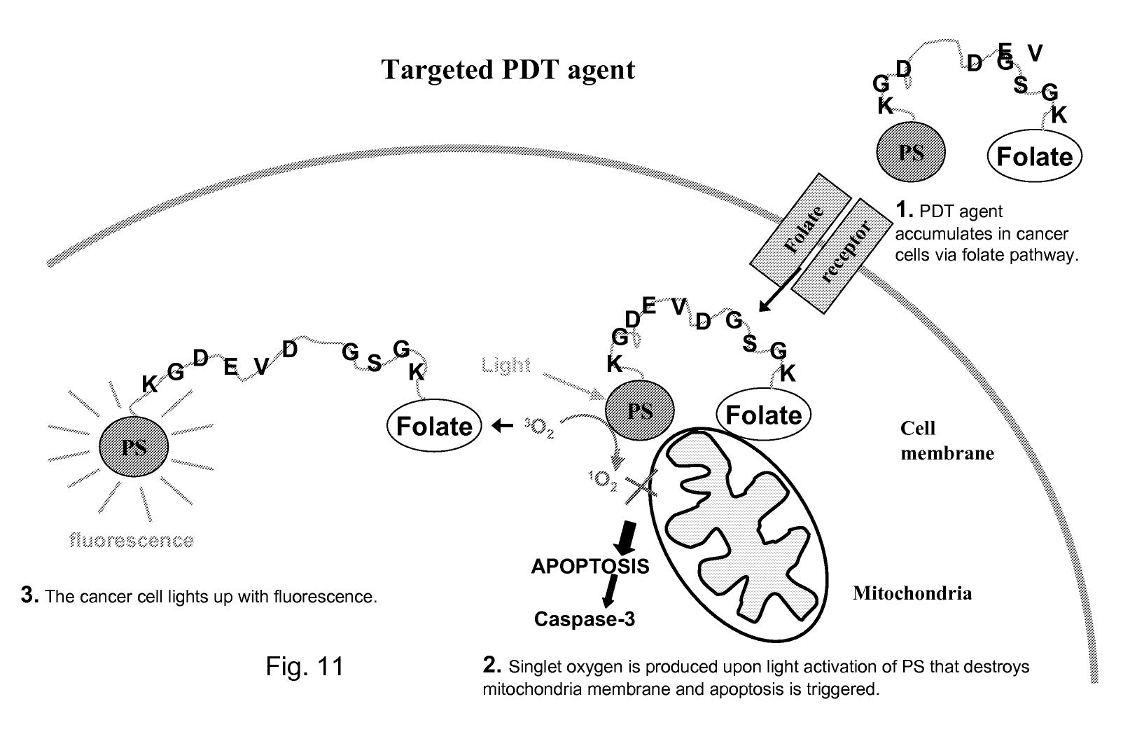

### DETAILED DESCRIPTION OF THE INVENTION

Terms are used herein as generally used in the art, unless otherwise defined. Conjugates

In an embodiment of the present invention, the conjugates of the present invention have following characteristics: 1) they contain a hydrophilic substrate; 2) they contain a photosensitizer (P) comprising a fluorophore; and 3) a targeting ligand. Upon PDT treatment, P generates ROS to kill the target cells.

### Substrates

As stated above, the conjugates of the present invention comprise a hydrophilic substrate, a photosensitizer comprising a fluorophore, and a targeting ligand. Substrates include but are not limited to polypeptides, nucleic acid molecules, synthetic polymers, galactose-containing compounds, or combinations thereof. The substrate serves multiple purposes: A) it is a stable and hydrophilic linker that prevents the separation of the targeting ligand and photosensitizer and enhances water solubility, B) it separates the photosensitizer from the targeting ligand to avoid the hindrance of targeting, C) it serves as a pharmacomodulator for better delivery efficiency and decreased normal tissue toxicity, and D) it is possible to exchange it with other peptide sequences for targeting subcellular organelles.

Two designations for amino acids are used interchangeably throughout this application, as is common practice in the art: Alanine=Ala (A); Arginine=Arg (R); Aspartic Acid=Asp (D); Asparagine=Asn (N); Cysteine=Cys (C); Glutamic Acid=Glu (E); Glutamine=Gln (Q); Glycine=Gly (G); Histidine=His (H); Isoleucine=Ile (I); Leucine=Leu (L); Lysine=Lys (K); Methionine=Met (M); Phenylalanine=Phe (F); Proline=Pro (P); Serine=Ser (S); Threonine=Thr (T); Tryptophan=Trp (W); Tyrosine=Tyr (Y); Valine=Val (V).

In another embodiment the substrate comprises a sequence selected from the group consisting of Asp-Glu-Val-Ile(SEQ ID NO: 1), Asp-Glu-Thr-Asp(SEQ ID NO: 2), Leu-Glu-His-Asp(SEQ ID NO: 3), Asp-Glu-His-Asp(SEQ ID NO: 4), Trp-Glu-His-Asp(SEQ ID NO: 5), Leu-Glu-Thr-Asp(SEQ ID NO: 6), Asp-Glu-Val-Asp(SEQ ID NO: 7), Val-Glu-His-Asp(SEQ ID NO: 8), and Ile-Glu-Ala-Asp(SEQ ID NO: 9).

In another embodiment, the substrate comprises a sequence selected from the group consisting of X-Asp-Glu-Val-Ile(SEQ ID NO: 1)-Y, X-Asp-Glu-Thr-Asp(SEQ ID NO: 2)-Y, X-Leu-Glu-His-Asp(SEQ ID NO: 3)-Y, X-Asp-Glu-His-Asp(SEQ ID NO: 4)-Y, X-Trp-Glu-His-Asp(SEQ ID NO: 5)-Y, X-Leu-Glu-Thr-Asp(SEQ ID NO: 6)-Y, X-Asp-Glu-Val-Asp(SEQ ID NO: 7)-Y, X-Val-Glu-His-Asp(SEQ ID NO: 8)-Y, and X-Ile-Glu-Ala-Asp(SEQ ID NO: 9)-Y, wherein X and Y are each independently a polypeptide comprising from one to about 15 amino acids. In another embodiment the substrate comprises the amino acid sequence Asp-Glu-Val-Asp(SEQ ID NO: 7). In yet another embodiment the substrate comprises the amino acid sequence Gly-Asp-Glu-Val-Asp-Gly-Ser-Gly-Lys (SEQ ID NO: 10). In yet another embodiment the substrate comprises the amino acid sequence Lys-Gly-Asp-Glu-Val-Asp-Gly-Ser-Gly-Lys (SEQ ID NO: 11).

In an embodiment of the present invention, when the photosensitizer is attached to X, the targeting ligand is attached to Y; and when the targeting ligand is attached to X, the photosensitizer is attached to Y. There can be at least from about 3 to about 10 substrate amino acids between the photosensitizer and the targeting ligand, from about 4 to about 8 substrate amino acids between the photosensitizer and the targeting ligand, or there are from about 5 to about 7 substrate amino acids between the photosensitizer and the targeting ligand.

In an aspect of the present invention, X and Y are each independently from 1 to about 25 amino acids, from 2 to about 15 amino acids, or from about 5 to about 10 amino acids in length.

In an additional embodiment, when the substrate is a polypeptide, when the photosensitizer is attached to the N-terminal amino acid of the polypeptide, the targeting ligand is attached to the C-terminal amino acid of the polypeptide; and when the photosensitizer is attached to the C-terminal amino acid of the polypeptide, the targeting ligand is attached to the N-terminal amino acid of the polypeptide.

In another embodiment the conjugate comprises 1) pyropheophorbide α as an imaging (NIR fluorescence) and therapeutic (photosensitizer) agent, 2) a caspase-3 substrate: GDEVDGSGK (SEQ ID NO: 10) or KGDEVDGSGK (SEQ ID NO: 11), and 3) folate as a cancer-specific delivery vehicle.

### Photosensitizer

As discussed previously, the conjugates of the present invention comprise a substrate, a photosensitizer, and a targeting ligand. As used herein, the term “photosensitizer” encompasses any agent used in photodynamic therapy. If not quenched, such agents become activated upon exposure to light and oxygen, producing lethal reactive oxygen species that kill, for example, tumor cells. Accordingly, an activated form of a photosensitizer produces lethal reactive oxygen species. In a one embodiment, the photosensitizers used in the present invention generate singlet oxygen upon exposure to oxygen and light of the appropriate wavelength. Photosensitizers can comprise a fluorophore. A fluorophore is any material capable of emitting fluorescence.

In an aspect of the present invention, the photosensitizer is a free base or metal complex of a compound selected from the group consisting of a porphyrin (e.g., porphyrin), a reduced porphyrin (e.g., chlorin), a chlorophyll, a chlorophyll derivative (e.g., phyropheophorbide, chlorin e6, chlorin p6 and purpurin 18), synthetic chlorin (e.g., a benzoporphyrin derivative and purpurin), bacteriochlorin (e.g., bacteriochlorophyll derivative, synthetic bacteriochlorin, porphyrin isomer (e.g., porphycence, heteroatom-fused porphyrin and inverted porphyrin), an expanded porphyrin (e.g., texaphyrin), and porphyrin analog (e.g., phthalocyanine and naphthalocyanine). In addition, the photosensitizer can be a nonporphyrin (e.g., hypericin, cationic dye (i.e., rhodamine), psoralen, and merocyanine 540).

Additional photosensitizers for use in the conjugates of the present invention will be apparent to one of skill in the art. As stated above, photosensitizers include but are not limited to those used in photodynamic therapy and those that have undergone or are currently undergoing clinical trials. For example, photosensitizers listed in Pandey, R. K. and G. Zheng, “Porphyrins as Photosensitizers in Photodynamic Therapy” in The Porphyrin Handbook, Kadish, K. M. et al., Eds., Academic Press (2000), which is hereby incorporated by reference in its entirety, can be used in the conjugates of the present invention.

### Targeting Ligands

As used herein, the term “targeting ligand” encompasses any agent which selectively binds to a cell or tissue to be treated with the conjugates of the invention. In another embodiment, targeting ligands selectively bind to tumor tissue or cells versus normal tissue or cells of the same type. In certain embodiments the targeting ligands general are ligands for cell surface receptors that are over-expressed in tumor tissue. Cell surface receptors over-expressed in cancer tissue versus normal tissue include, but are not limited to epidermal growth factor receptor (EGFR) (overexpressed in anaplastic thyroid cancer and breast and lung tumors), metastin receptor (overexpressed in papillary thyroid cancer), ErbB family receptor tyrosine kinases (overexpressed in a significant subset of breast cancers), human epidermal growth factor receptor-2 (Her2/neu) (overexpressed in breast cancers), tyrosine kinase-18-receptor (c-Kit) (overexpressed in sarcomatoid renal carcinomas), HGF receptor c-Met (overexpressed in esophageal adenocarcinoma), CXCR4 and CCR7 (overexpressed in breast cancer), endothelin-A receptor (overexpressed in prostate cancer), peroxisome proliferator activated receptor delta (PPAR-delta) (overexpressed in most colorectal cancer tumors), PDGFR A (overexpressed in ovarian carcinomas), BAG-1 (overexpressed in various lung cancers), soluble type II TGF beta receptor (overexpressed in pancreatic cancer) folate and integrin (e.g. αvβ3).

The folate receptor is a glycosylphosphatidylinositol-anchoredglycoprotein with high affinity for the vitamin folic acid (Kd˜10⁻⁹ M) (Leamon, C. P. et al., Biochemical Journal. 1993 May 1; 291 (Pt. 3):855-60). Folate receptor has been identified as a tumor-marker, which is expressed at elevated levels relative to normal tissues on epithelial malignancies, such as ovarian, colorectal, and breast cancer (Wang, S. et al., Journal of Controlled Release. 1998 Apr. 30; 53(1-3):39-48). It has been shown that when folate is covalently linked to either a single molecule or assembly of molecules via its γ-carboxyl moiety, its affinity for the cell surface receptors remains essentially unaltered. Following endocytosis and vesicular trafficking, much of the material is released into the cell cytoplasm. The folate receptor may then recycle to the cell surface; thus, each folate receptor may bring many folate conjugates into the cell.

In another embodiment, the targeting ligand is a cell surface receptor ligand for a receptor selected from the group consisting of folate, Her-2/neu, integrin, EGFR, metastin, ErbB, c-Kit, c-Met, CXR4, CCR7, endothelin-A, PPAR-delta, PDGFR A, BAG-1, and TGF beta. In yet another embodiment, the targeting ligand is a cell surface receptor ligand for folate receptor.

The targeting ligand can be covalently linked to the substrate or the photosensitizer as long as the targeting ligand is not linked in such a manner so that interference with binding of the targeting ligand to its receptor does not occur. In one embodiment, the photosensitizer and the targeting ligand are covalently linked to opposite ends of the substrate.

### Methods of Treatment

The present invention is directed to a method of inhibiting the growth of cancer cells, in vitro or in vivo, comprising the steps of contacting the cancer cells with a conjugate of the present invention, and exposing the cancer cells to an effective amount of artificial irradiation. In one aspect, the invention provides methods of inhibiting the growth of cancer cells, such as breast, lung, pancreas, bladder, ovarian, testicular, prostate, retinoblastoma, Wilm's tumor, adrenocarcinoma or melanoma. In one embodiment, the cancer tumor is a prostate cancer tumor.

The present invention provides a method for selectively killing tumor cells expressing a target that specifically binds to the targeting ligand. For example, this invention provides a method of treating carcinomas (for example human carcinomas) in vivo. This method comprises the steps of administering to a subject a pharmaceutically effective amount of a composition containing at least one of the conjugates of the present invention.

In accordance with the practice of this invention, the subject may be a human, equine, porcine, bovine, murine, canine, feline, and avian subjects. Other warm blooded animals are also included with the scope of this invention.

The present invention also provides a method for treating a subject suffering from cancer. The subject may be a human, dog, cat, mouse, rat, rabbit, horse, goat, sheep, cow, chicken. The cancer may be identified as a breast, lung, pancreas, bladder, ovarian, testicular, prostate, retinoblastoma, Wilm's tumor, adrenocarcinoma or melonoma and is generally characterized as a group of cells which over-express and/or have an over-abundance of the target. This method comprises the steps of administering to the subject a cancer killing amount of one or more conjugates of the present invention.

Also provided is a method of inhibiting the proliferation of mammalian tumor cells which comprises the steps of contacting the mammalian tumor cells with a sufficient concentration of the conjugate of the invention, and exposing the mammalian tumor cells to artificial irradiation.

The subject invention further provides methods for inhibiting the growth of human tumor cells, treating a tumor in a subject, and treating a proliferative-type disease in a subject. These methods comprise the steps of administering to the subject an effective amount of the conjugate of the invention.

The present invention also provides for a method of treating a disease state comprising administering to a target tissue of a patient a conjugate of the present invention and irradiating the photosensitizer thereby killing the target tissue.

It is apparent that the present invention encompasses pharmaceutical compositions, combinations and methods for treating human carcinomas. For example, the invention includes pharmaceutical compositions for use in the treatment of human carcinomas comprising a pharmaceutically effective amount of the conjugate of the present invention and a pharmaceutically acceptable carrier.

The compositions may additionally include other drugs or antibodies treating carcinomas.

The conjugates of the invention can be administered using conventional modes of administration including, but not limited to, intravenous, intraperitoneal, oral, intralymphatic, or administration directly into the tumor.

The conjugates of the invention may be provided in a variety of dosage forms which include, but are not limited to, liquid solutions or suspension, tablets, pills, powders, suppositories, polymeric microcapsules or microvesicles, liposomes, and injectable or infusible solutions as well as conjugates of the above with polyethylene glycol (pegylated carriers). The form depends upon, among other things, the mode of administration and the therapeutic application.

The compositions of the invention also include conventional pharmaceutically acceptable carriers and adjuvants known in the art such as human serum albumin, ion exchangers, alumina, lecithin, buffer substances such as phosphates, glycine, sorbic acid, potassium sorbate, and salts or electrolytes such as protamine sulfate.

The most effective mode of administration and dosage regimen for the compositions of this invention depends upon the severity and course of the disease, the patient's health and response to treatment and the judgment of the treating physician. Accordingly, the dosages of the compositions should be titrated to the individual patient. Nevertheless, an effective dose of the compositions of this invention may be in the range of from about 1 to about 2000 mg/kg. The dosage can also be from about 2 to about 1000 mg/kg, about 4 to about 400 mg/kg, or about 5 to about 100 mg/kg.

The interrelationship of dosages for animals of various sizes and species and humans based on mg/kg of surface area is described by Freireich, E. J. et al., Cancer Chemother. 50 (4): 219-244 (1966). Adjustments in the dosage regimen may be made to optimize the tumor cell growth inhibiting and killing response, e.g., doses may be divided and administered on a daily basis or the dose reduced proportionally depending upon the situation (e.g., several divided doses may be administered daily or proportionally reduced depending on the specific therapeutic situation.

The dose of the composition of the invention required to achieve cures may be further reduced with schedule optimization.

In accordance with the practice of the invention, the pharmaceutical carrier may be a lipid carrier or lipoprotein particle such as LDL, HDL, VLDL, IDL or chylomicron. The lipid carrier may be a phospholipid. Further, the lipid carrier may be a fatty acid. Also, the lipid carrier may be a detergent. As used herein, a detergent is any substance that alters the surface tension of a liquid, generally lowering it.

In one example of the invention, the detergent may be a nonionic detergent. Examples of nonionic detergents include, but are not limited to, polysorbate 80 (also known as Tween 80 or (polyoxyethylenesorbitan monooleate), Brij, and Triton (for example Triton WR-1339 and Triton A-20).

Alternatively, the detergent may be an ionic detergent. An example of an ionic detergent includes, but is not limited to, alkyltrimethylammonium bromide.

Additionally, in accordance with the invention, the lipid carrier may be a liposome or polymerosome as well as conjugates of the above with polyethylene glycol (pegylated carriers). As used in this application, a “liposome” is any membrane bound vesicle which contains any molecules of the invention or combinations thereof.

The human is preferentially exposed to artificial irradiation is selected from the group consisting of artificial ultraviolet, infrared (IR), gamma-irradiation, x-ray and visible light. In certain embodiments the irradiation is IR or near-infrared (NIR). The artificial irradiation can be applied about 5 minutes to about 3 hours after administering the conjugate of the present invention or the artificial irradiation is applied about 10 to about 60 minutes after administering the conjugate of the present invention.

The amount of conjugate administered in the formulation will depend upon the photosensitizer chosen. The amount of conjugate administered can be about 0.1 to about 10.0 mg/kg body weight of the subject, about 0.3 to about 6 mg/kg body weight, or about 0.4 to about 4.0 mg/kg body weight.

In another embodiment, in the methods of treating cancer of the present invention, the artificial irradiation can be applied for about 10 seconds to about 60 minutes, or the artificial irradiation is applied for about 15 seconds to about 30 minutes.

In yet another embodiment, the present invention further provides pharmaceutical compositions which comprise the conjugates of the present invention and a pharmaceutically acceptable carrier.

The present invention further provides a method for treating cancer in a subject cancer comprising the steps of administering a therapeutically effective amount of the pharmaceutical composition of the present invention.

The present invention further provides a method for treating viral infections in a subject, comprising the steps of administering a therapeutically effective amount of the pharmaceutical composition of the present invention.

The examples below explain the invention in more detail. The following preparations and examples are given to enable those skilled in the art to more clearly understand and to practice the present invention. The present invention, however, is not limited in scope by the exemplified embodiments, which are intended as illustrations of aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### EXAMPLES

### Example 1

### Overview of Examples

Pyro-GDEVDGSGK (PP) Pyro-GDEVDGSGK-Folate (PPF) were purified by HPLC (Waters 600 Controller with a 2996 Photodiode Array Detector). The eluents were A=0.1M TEAA (pH 7) and B=acetonitrile and the method used for all injections was 90% of A and 10% of B to 100% of B in 45 minutes with flow 1.5 mL/min on a Zorbax 300SB-C3 column The coupling reagents HBTU (O-(Benzotriazol-1-yl)-N,N,N′,N′-tetramethyluronium hexafluorophosphate) and HOBt (1-Hydroxybenzotriazole) were purchased from Fluka and ACROS respectively. MALDI-ToF was measured on an Applied Biosystems Voyager DE Mass Spectrometer and fluorescence was measured on a Perkin Elmer LS50B Luminescence Spectrometer. We acquired confocal images using a Leica TCS SP2 Confocal Microscope and Xenogen images at the Bioluminescence Molecular Imaging Core facility at UPenn on an IVIS Xenogen Imager. KB cells (human epidermal cancer cells; folate receptor positive), HT 1080 cells (human fibrosarcoma cells; folate receptor negative), and CHO (Chinese hamster ovary cells; folate receptor negative) were purchased from ATCC. “Complete MEM” (for HT 1080 and KB cells) consists of 85% Minimum Essential Medium (MEM), 10% Fetal Bovine Serum, 2% of 1.5 g/L Sodium Bicarbonate, 1% of 200 mM L-glutamine, 1% of 0.1 mM Non-essential Amino Acids, and 1% of 1 mM Sodium Pyruvate and “MEM containing 0.8% BSA” consists of MEM supplemented with 8 g/L of Bovine Serum Albumin “Complete F12K” (for CHO cells) consists of 87% Ham's F12K medium, (F12K), 10% Fetal Bovine Serum, 2% of 1.5 g/L Sodium Bicarbonate and 1% of 200 mM Lglutamine and “F12K containing 0.8% BSA” consists of F12K supplemented with 8 g/L of Bovine Serum Albumin PP, PPF, and Pyro-K-Folate (PKF) prepared for in vivo experiments were dissolved in 1 μL DMSO and 20 μL PBS, filtered through a 0.22 μm filter and further diluted with sterile saline to the 120-150 μL volume. For the in vitro experiments, PPF and PKF were first dissolved in DMSO (no more than 0.5% of total volume) then diluted with 0.1% Tween-80 in DNA-water, filtered through a 0.22 μm filter and further diluted with MEM containing 0.8% BSA (HT 1080 and KB cells) or F12K containing 0.8% BSA (CHO cells). The laser for PDT treatment was tuned to 670 nm with a fluence rate of 20 mW/cm2. Mice were euthanized according to guidelines established by the Institutional Animal Care and Use Committee of the University of Pennsylvania.

### Example 2

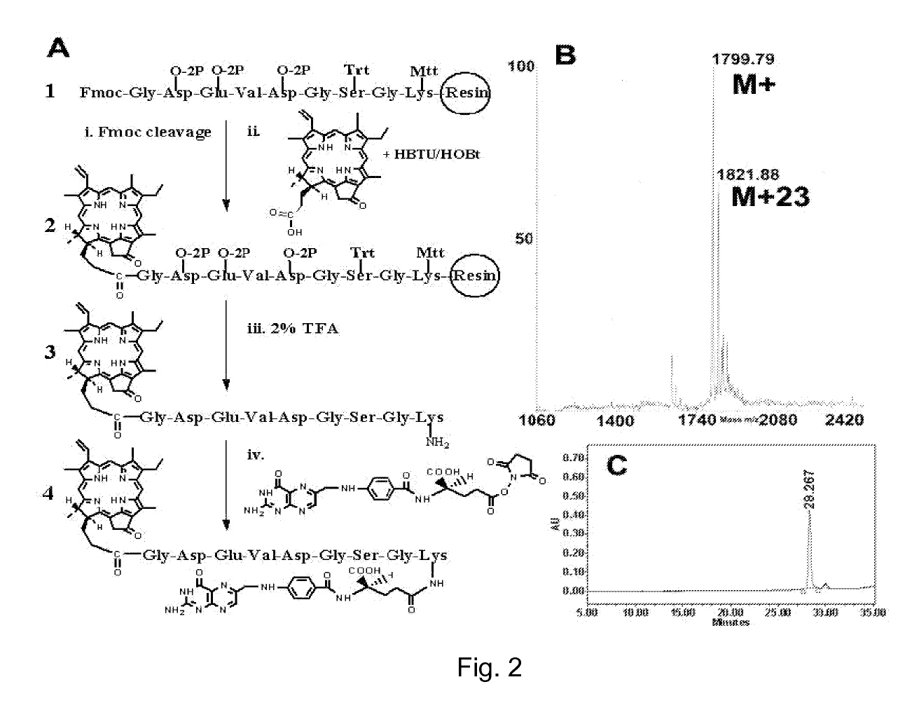

Synthesis of PPF. The peptide Fmoc-GD(O-2-PhiPr)E(O-2-PhiPr)VD(O-2-PhiPr)GS(Trt)GK(Mtt) was synthesized by manual SPPS using commercially available Fmoc amine protected amino acids as building blocks and Sieber resin (cleavable by 2% TFA) as a solid phase, all purchased from Novabiochem®. After the last Fmoc group cleavage, the resin was washed with NMP and Pyro-acid was coupled to the N-terminal glycine to give Pyro-GD(0-2-PhiPr)E(O-2-PhiPr)VD(O-2-PhiPr)GS(Trt)GK(Mtt)-Sieber resin. The molar ratio of Pyro/HOBt/HBTU to the peptide was 3/3/3:1 and the reaction was done in a shake flask with a 12 hour coupling time using dry NMP as a solvent. The resin was then washed with NMP, capped by 0.3 M Acetylimidazol in NMP for 15 minutes and washed again by an excess of NMP, DCM and dry methanol, transferred from the shake flask and dried. The compound (37.7 mg, 11.5 mmol) was cleaved from the resin and deprotected in one step by 2% TFA/5% TIS/DCM for 1 hour to yield Pyro-GDEVDGSGK(NH2) with the ε-NH2 group of the C-terminal lysine exposed. The compound was precipitated from the cleavage solution by dry ether and pre-purified by a few cycles of ether precipitation-DMSO dissolving. PP (10.9 mg, 6.8 mmol) was dried under high vacuum and, without further purification, used in the next reaction. For in vivo experiments, PP was purified by HPLC. The structure was confirmed by MALDI-ToF (mass calculated: 1377.64, found: 1377.95). Crude PP (9.3 mg, 5.8 mmol) was dissolved in 100 μl of dry 1% DIPEA/DMSO and reacted for 2 hours with Folate-NHS (4.7 mg, 6 mmol) dissolved in 100 μl of dry DMSO to give Pyro-GDEVDGSGK-Folate (PPF). The reaction was concentrated by precipitation with ether and directly separated by HPLC. Purified PPF (11.7 mg, 5.5 μmol) was dried under high vacuum and stored at −20° C. The purity was checked by HPLC and MALDI-ToF (mass calculated: 1800.76, found: 1799.79, FIG. 2B).

### Example 3

Confocal microscopy. KB and HT 1080 cells were grown in 4-well Lab-Tek chamber slides (Naperville, Ill.) at a density of 50,000 cells/well and grown for 24 hours in complete MEM, then rinsed with HBSS (Hank's balanced salt solution) and incubated with 50 μM PPF in 300 μl of MEM containing 0.8% BSA at 37° C. for 5 hours. Cells were repeatedly rinsed with HBSS after the incubation and fixed by 1% formaldehyde in PBS for 20 minutes prior to scanning with confocal microscopy. The settings for confocal microscopy were as follows: 640-800 nm detector slit for detection of Pyro fluorescence, with zoom=2, expander=3, resolution 512×512, 633 nm excitation wavelength, 40× objective, 100% power, 851.1 gain, and −28.5 offset.

### Example 4

Flow cytometry experiments. KB, HT 1080, and CHO cells were seeded in T25 flasks (each flask 106 cells) and grown for 1 day in complete MEM (KB cells and HT 1080 cells) or complete F12K (CHO cells) medium. The cells were incubated for 12 hours with 5 μM PPF or PKF in MEM containing 0.8% BSA (KB and HT 1080 cells) or F12K containing 0.8% BSA (CHO cells) 1.3 mL/flask alone or with 5 mM folic acid added, and at the end of incubation the medium was aspirated and cells washed 3 times with 2 mL of PBS. The cells were detached by 0.25% Trypsin-EDTA, fixed by 1% formaldehyde (methanol free) in PBS, resuspended in 1 mL of PBS and immediately analyzed on a BD LSRII machine at the Flow Cytometry Laboratory, Abramson Cancer Center at the University of Pennsylvania focusing on Pyro fluorescence (exc. 633 nm, em. 695 nm).

### Example 5

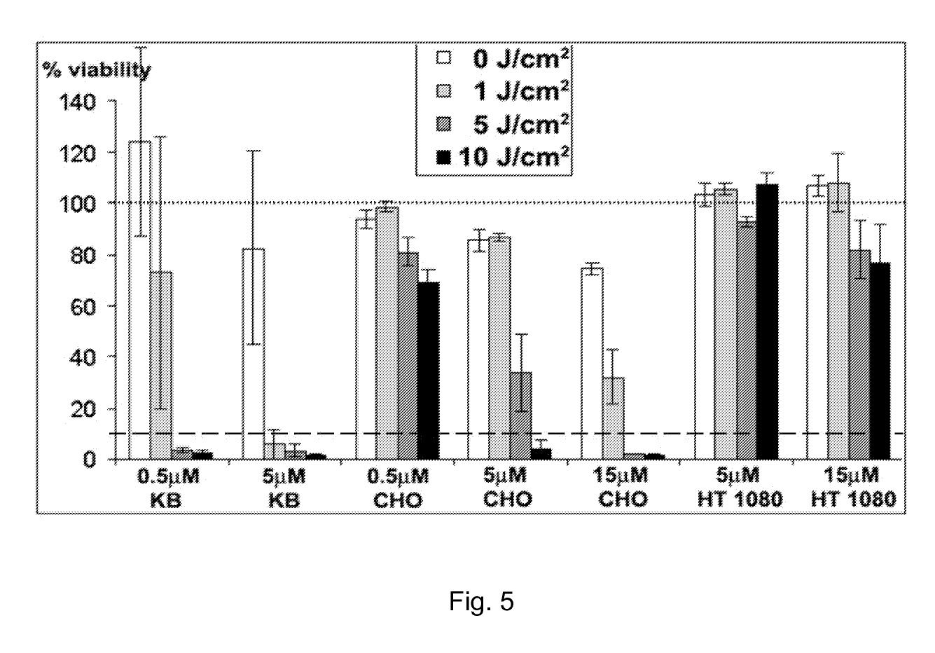

Cell viability (MTT) assay. KB, HT 1080, and CHO cells were seeded in clear 96-well plates at a density of 50,000 cells/well in 250 μl of complete MEM (KB and HT 1080 cells) or complete F12K (CHO cells) and grown for 24 hours at 37° C. Cells were subsequently rinsed with HBSS and incubated with no or 0.5, 5 or 15 μM PPF in MEM containing 0.8% BSA (KB and HT 1080 cells) or F12K containing 0.8% BSA (CHO cells) for 24 hours. Cells were then rinsed with HBSS, 100 μl/well of complete MEM was added, and the cells were either kept in the dark or treated with three different light doses (1, 5, and 10 J/cm2) by a 670 nm laser with 20 mW/cm2 fluence rate. After incubation for 24 hours at 37° C., the cells were incubated for 2 hours with a 0.5 mg/mL solution of MTT in complete MEM/complete F12K, that was disposed afterwards and replaced with 100 μL, of equal parts of DMSO and 70% isopropanol in 0.1M HCl. Absorbance at 570 nm was measured. Data shown in FIG. 5 are based on 3-4 different experiments and the results were expressed as mean±standard error.

### Example 6

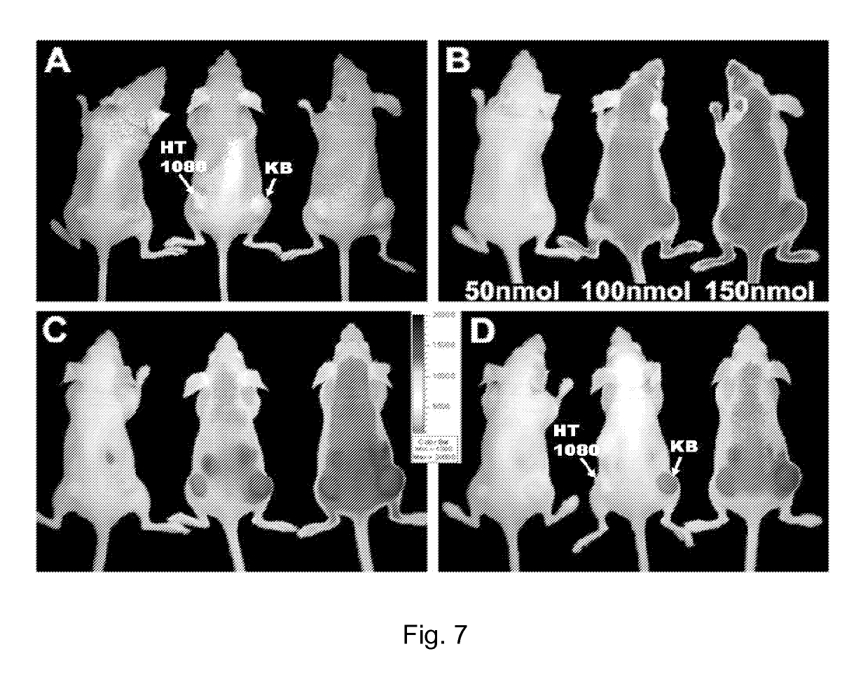

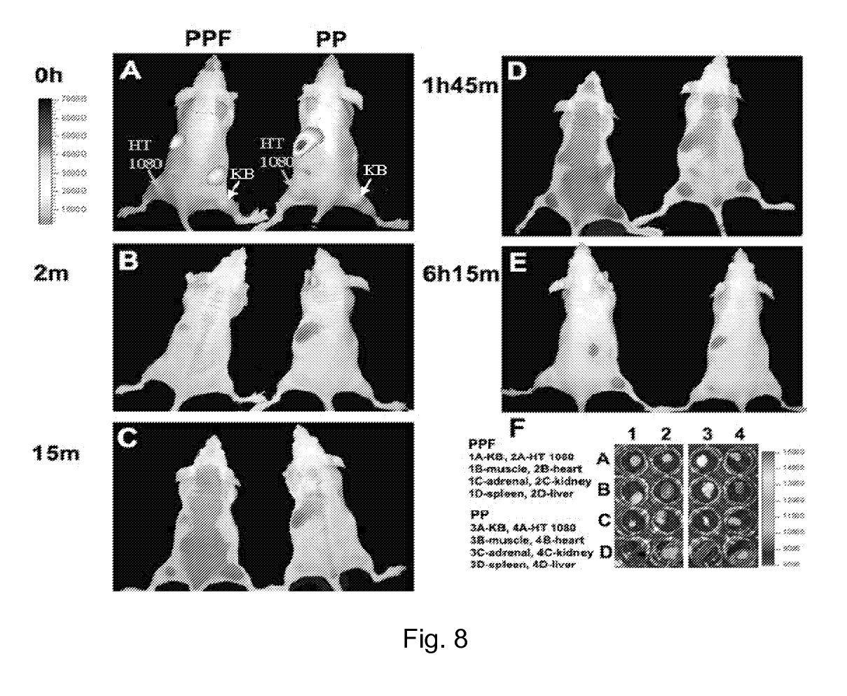

In vivo imaging. Nude mice were inoculated subcutaneously with 107 KB cells above the right leg and 107 HT 1080 cells above the left leg and the tumors were grown for about 7 days. FIG. 7: Three mice were first scanned with a Xenogen IVIS imager with a Cy 5.5 filter (λexc=615-665 nm, λem=695-770 nm) and then intravenously injected with 50 nmol (left), 100 nmol (middle), and 150 nmol (right mouse) of PPF 60-180 μl/mouse into the tail vein. The mice were imaged 5 minutes, 6 hours, and 24 hours after injection. Two double tumor mice were intravenously injected with 30 nmol of PPF (left mouse) and 30 nmol PP (right mouse) and imaged. The mice were sacrificed 26-30 hours after the injection and the organs (KB tumor, HT 1080 tumor, muscle, heart, adrenal, kidney, spleen, and liver) were imaged directly after dissection and used for biodistribution. See also FIG. 8.

### Example 7

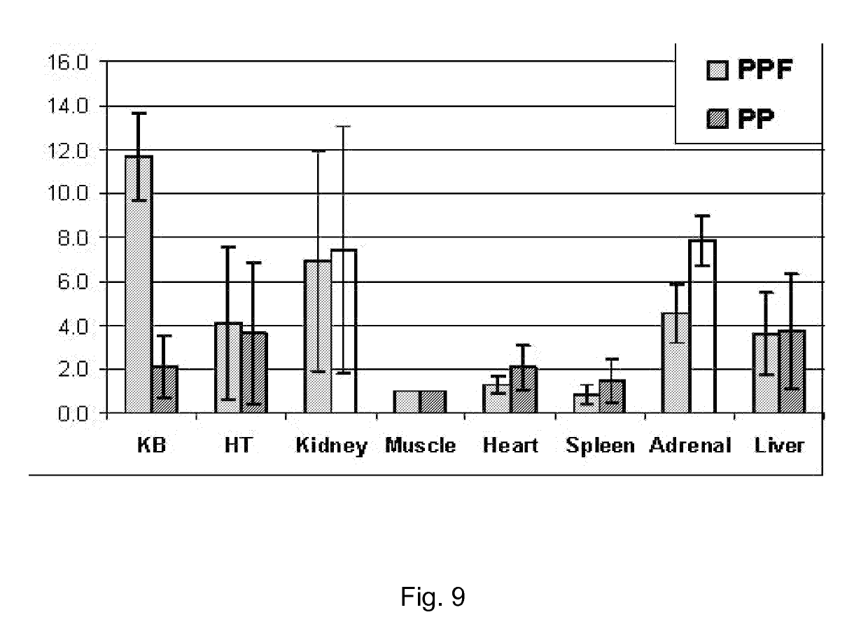

Ex vivo organ biodistribution. The mice were euthanized 26-30 hours after drug injection and the dissected organs were washed in PBS and scanned by Xenogen in a clear 24-well plate. The organs were weighed and disintegrated in DMSO:MeOH (2:3). After settling, the fluorescence was measured (excitation 410 nm, emission 670 nm) and divided by weight for each organ. This ratio of fluorescence vs. weight was normalized with respect to muscle and plotted in FIG. 9.

The entire disclosure of all publications (including patents, patent applications, journal articles, laboratory manuals, books, or other documents) cited herein are hereby incorporated by reference. Notwithstanding such incorporation, applicants do not regard the scope of the terms used herein as limited by their usage in the cited publications.

What is claimed is:

1. A conjugate comprising a substrate, a photosensitizer comprising a fluorophore, and a targeting ligand, wherein said photosensitizer and said targeting ligand are covalently linked to said substrate.

2. The conjugate of claim 1, wherein said photosensitizer and said targeting ligand are attached at opposite ends of said substrate.

3. The conjugate of claim 2, wherein said substrate is a polypeptide.

4. The conjugate of claim 3, wherein said substrate comprises a polypeptide and if said photosensitizer is attached to the N-terminal amino acid of said polypeptide, then said targeting ligand is attached to the C-terminal amino acid of said polypeptide; and if said photosensitizer is attached to the C-terminal amino acid of said polypeptide, then said targeting ligand is attached to the N-terminal amino acid of said polypeptide.

5. The conjugate of claim 4, wherein said substrate comprises an amino acid sequence selected from the group consisting of Asp-Glu-Val-Ile(SEQ ID NO: 1), Asp-Glu-Thr-Asp(SEQ ID NO: 2), Leu-Glu-His-Asp(SEQ ID NO: 3), Asp-Glu-His-Asp(SEQ ID NO: 4), Trp-Glu-His-Asp(SEQ ID NO: 5), Leu-Glu-Thr-Asp(SEQ ID NO: 6), Asp-Glu-Val-Asp(SEQ ID NO: 7), Val-Glu-His-Asp(SEQ ID NO: 8), and Ile-Glu-Ala-Asp(SEQ ID NO: 9).

6. The conjugate of claim 5, wherein said substrate comprises the amino acid sequence Asp-Glu-Val-Asp(SEQ ID NO: 7).

7. The conjugate of claim 6, wherein said substrate comprises the amino acid sequence Gly-Asp-Glu-Val-Asp-Gly-Ser-Gly-Lys (SEQ ID NO: 10).

8. The conjugate of claim 7, wherein said substrate comprises the amino acid sequence Lys-Gly-Asp-Glu-Val-Asp-Gly-Ser-Gly-Lys (SEQ ID NO: 11).

9. The conjugate of claim 5, wherein said substrate comprises an amino acid sequence selected from the group consisting of X-Asp-Glu-Val-Ile(SEQ ID NO: 1)-Y, X-Asp-Glu-Thr-Asp(SEQ ID NO: 2)-Y, X-Leu-Glu-His-Asp(SEQ ID NO: 3)-Y, X-Asp-Glu-His-Asp(SEQ ID NO: 4)-Y, X-Trp-Glu-His-Asp(SEQ ID NO: 5)-Y, X-Leu-Glu-Thr-Asp(SEQ ID NO: 6)-Y, X-Asp-Glu-Val-Asp(SEQ ID NO: 7)-Y, X-Val-Glu-His-Asp(SEQ ID NO: 8)-Y, and X-Ile-Glu-Ala-Asp(SEQ ID NO: 9)-Y, wherein X and Y are each independently a polypeptide comprising from one to about 15 amino acids.

10. The conjugate of claim 9, wherein if said photosensitizer is attached to said X, then said targeting ligand is attached to said Y; and if said targeting ligand is attached to said X, then said photosensitizer is attached to said Y.

11. The conjugate of claim 1, wherein said photosensitizer is a free base or metal complex of a compound selected from the group consisting of a pyropheophorbide, a purpurin, a porphyrin, a chlorin, a bacteriochlorin, a phthalocyanine, a naphthalocyanine, a hypericin, a porphyrin isomer, an expanded porphyrin, a cationic dye, a psoralen, and a merocyanine 540.

12. The conjugate of claim 11, wherein said expanded porphyrin is texaphyrin.

13. The conjugate of claim 1, wherein said targeting ligand is a ligand for a cell surface receptor.

14. The targeting ligand of claim 13, wherein said cell surface receptor is selected from the group consisting of folate, Her-2/neu, integrin, EGFR, metastin, ErbB, c-Kit, c-Met, CXR4, CCR7, endothelin-A, PPAR-delta, PDGFR A, BAG-1, and TGF beta receptors.

15. The targeting ligand of claim 14, wherein said cell surface receptor is folate receptor.

16. A method of inhibiting the growth of a cancer cell comprising:
(a) contacting said cancer cell with the conjugate of claim 1 or 15; and
(b) exposing said cancer cell to an effective amount of artificial irradiation.

17. The method of claim 16, wherein said cancer cell is selected from the group consisting of prostate, breast, ovarian, lung, pancreatic, bladder, testicular, retinoblastoma, Wilm's tumor, adrenocarcinoma and or melonoma cells.

18. The method of claim 16, wherein said artificial irradiation is selected from the group consisting of artificial ultraviolet, near-infrared, infrared, gamma-irradiation, x-ray and visible light.

19. The method of claim 16, wherein said artificial irradiation is applied at a wavelength ranging from about 20 nm less than the maximum absorption of the photosensitizer to about 20 nm greater than the maximum absorption of the photosensitizer.

20. The method of claim 16, wherein said artificial irradiation is applied at a rate of about 10 to about 150 mW/cm².

21. The method of claim 20, wherein said artificial irradiation is applied at a rate of about 35 to about 100 mW/cm².

22. The method of claim 21, wherein said artificial irradiation is applied at a rate of about 75 mW/cm².

23. A pharmaceutical composition comprising the conjugate of claim 1 or 15.

24. A method for the treatment of cancer in a subject comprising administering a therapeutically effective amount of the pharmaceutical composition of claim 23 to a subject in need thereof.
